(19) United States
(12) Patent Application Publication
(10) Pub. No.: US 2026/0021252 A1
(43) Pub. Date: Jan. 22, 2026

(54) MEDICAMENT DELIVERY ASSEMBLY

(71) Applicant: Owen Mumford Limited, Woodstock, Oxfordshire (GB)

(72) Inventor: Toby Ken Cowe, Woodstock, Oxfordshire (GB)

(21) Appl. No.: 19/119,638

(22) PCT Filed: Oct. 11, 2022
(86) PCT No.: PCT/EP2022/078225

§ 371 Date: Apr. 9, 2025

(30) Foreign Application Priority Data
Oct. 10, 2022 (GB) 2214868.8

(51) Int. Cl. A61M 5/24; A61M 5/00; A61M 5/31; A61M 5/32

(52) U.S. Cl. CPC A61M 5/24; A61M 5/002; A61M 5/3243; A61M 2005/3118

(57) ABSTRACT

A medicament delivery assembly for installation on a cartridge having a medicament chamber for containing a medicament; the medicament delivery assembly comprising a body for mounting on the cartridge; a hub comprising engagement means moveable from a first configuration where the resilient member is held in engagement with the body and a second configuration where the hub is moveable relative to the body; and a cannula mounted on the hub.

### TECHNICAL FIELD

The present disclosure relates to a medicament delivery assembly.

### BACKGROUND

Medicaments for subcutaneous injection are used in therapy in various different clinical situations, and a corresponding variety of injection devices have been developed to suit each situation.

For example, injectable medicaments may be supplied in traditional vials for use with a separate syringe. Such vials may be closed with a polymeric membrane or septum that can be pierced by a syringe needle, allowing the medicament to be drawn into the syringe. The septum is typically self-sealing to prevent leakage of the medicament once the needle is withdrawn.

Another approach is to provide a pre-filled, disposable syringe containing a single dose of the medicament. In this case, the medicament is contained in a tubular syringe body by a slidable stopper. In one common staked-needle design, sold under the registered trade mark Hypak (Becton Dickinson, New Jersey, USA), the needle is permanently fixed to the distal end of the syringe body. In other designs, the syringe body may be configured to accept a removable needle, for instance with a luer-type fitting. A plunger protrudes from a proximal end of the syringe body to allow the stopper to be displaced for delivery of the medicament through the needle. These disposable devices are relatively inexpensive and are often more convenient than a traditional syringe and vial for delivery of a fixed dose of medicament but, like the syringe and vial, they are generally suitable for use only by trained individuals with a reasonable degree of manual dexterity. Accordingly, more sophisticated injection devices have been developed for use in situations where some degree of automation of the injection process is desired and/or where the use of a traditional syringe or pre-filled syringe would be difficult or inconvenient. Examples include the self-administration of medicaments by patients and emergency treatment by untrained personnel.

For example, injection devices known as auto-injectors are generally intended for the administration of a single, fixed dose of medicament. Typically, in such devices, one or more of needle insertion, medicament delivery, dose indication, needle retraction and deployment of a needle shield after injection are triggered by one or more user operations, such as operating a trigger button or slider.

The medicament dose in an auto-injector is usually provided in the form of a disposable, pre-filled glass syringe with a fixed needle, such as a Hypak syringe of the type described above. Auto-injector mechanisms typically include a mechanically-driven plunger to drive the stopper of the syringe, and may be arranged to move the syringe body with respect to a body of the auto-injector to effect insertion and/or retraction of the needle. In many cases, the auto-injector is not re-usable and the pre-filled syringe cannot be replaced. Instead, when the medicament has been delivered, the auto-injector is discarded along with the pre-filled syringe within.

Other injection devices, sometimes known as injection pens, contain multiple doses of a medicament and are intended to be re-used. Re-usable devices are invariably designed for use with disposable, single-use double-ended needles, which are interchangeable to allow a fresh, sterile needle to be fitted for each injection. Such devices often include a mechanism for adjusting the amount of medicament to be delivered with each injection, and may incorporate a trigger mechanism to deliver automatically the required dose upon activation of a trigger button. Injection pen devices are routinely used for self-administration of, for example, insulin and human growth hormone.

In an injection pen, the medicament is typically packaged in a pre-filled cartridge. The medicament is contained between a self-sealing polymeric membrane or septum at the distal end of the cartridge, and a slidable stopper that can be driven by a plunger of the injection pen. The septum is pierced by the inside end of the needle when the needle is fitted to the device and, upon removal of the needle, the septum re-seals to prevent leakage of the medicament.

In some cases, the distal end of the cartridge includes a fitting that protrudes from the distal end of the injection pen device, and the needle engages directly with the cartridge. In other designs, the needle engages with the body of the injection pen device, rather than the cartridge itself. Most commonly, injection pen devices use screw-fit needles, although other needle designs, such as bayonet fittings and clip fittings, are also known.

Some injection pen devices employ replaceable cartridges, which allow the device to be refilled once each cartridge is empty. In other cases, the medicament cartridge is not replaceable, and the device is discarded when the medicament has been exhausted.

In some therapies, the administration of medicaments by infusion over a relatively long time period is preferred. One increasingly common example is the use of insulin pumps for the treatment of diabetes mellitus. In such cases, the medicament is typically packaged in a reservoir. The reservoir includes a slidable stopper that can be driven by a plunger mechanism in the pump, and a fitting to allow connection to a cannula. The fitting may include a luer-type or proprietary connection, and the cannula may be connected to the reservoir either by way of a flexible tube or, in so-called “patch pump” designs, directly or through a rigid connection. Some pump designs use refillable reservoirs, in which case the medicament may be supplied in a vial with a corresponding fitting to allow secure attachment of the pump reservoir during refilling. Other pump devices use pre-filled, cartridge-type reservoirs which are exchanged when empty.

It will be appreciated that, in all of these examples, the design of the primary packaging (i.e. the type of container in which a medicament is initially supplied from the manufacturer) is of key importance in the successful and safe operation of the injection or infusion device.

To ensure compatibility and safety of primary packages, it is necessary to test and validate every combination of medicament and primary packaging that is released to the market. The validation process may involve clinical trials and regulatory approval, and may therefore be extremely expensive and time-consuming.

As a result, many medicaments are currently available in only one or two different primary packages, reducing the choice of device available for administration of that medicament. In some cases, the cost of validating a new primary package for an existing medicament may be prohibitive. In other cases, where a new dedicated primary package for a new device is developed, the increased medicament cost may discourage the approval of new devices by healthcare funding bodies or insurers. Conversely, when designing new delivery devices for established medicaments, aspects of the design may be undesirably restricted by the type of primary packaging already available for the particular medicament in question.

### SUMMARY OF THE INVENTION

Aspects and embodiments of the invention provide a medicament delivery assembly and a medicament packaging assembly as claimed in the appended claims.

According to an aspect of the present invention there is provided a medicament delivery assembly for installation on a cartridge having a medicament chamber for containing a medicament; the medicament delivery assembly comprising a body for mounting on the cartridge a hub comprising engagement means moveable from a first configuration where the resilient member is held in engagement with the body and a second configuration where the hub is moveable relative to the body; and a cannula mounted on the hub.

Optionally the engagement means is further moveable to a third configuration where the engagement means prevents movement relative to the body.

The engagement means may be a resilient member. Optionally, the hub comprises a hub body and the engagement means comprises a projection extending distally from the hub body. The distally extending projection may further comprise a proximal outward extension spaced from the hub body and the distal end of the body comprises an annular flange which, in the first configuration is received between the hub body and the proximal outward extension. This holds the body and hub body to be held in the first configuration.

In a third configuration the distal end of the resilient member engages with a proximal surface of an annular flange on the distal end of the body.

The distal end of the resilient member may include an outward projection. Optionally, the hub includes a central portion through which the cannula is mounted. The engagement means are spaced away from the central portion to allow for inward flexion of the resilient member towards the central portion.

Optionally, in the second configuration the resilient member is flexed towards the hub body. Flexing the resilient member inwards disengages the annular flange at the distal end of the body and the proximal outward extension on the resilient member allowing relative movement between the hub body and the body.

The body includes an inwardly extending annular flange on its proximal end to engage with an annular flange on the distal end of a cartridge. The body may also include a proximal portion with a plurality of annular projections from its inner surface to engage with the outer surface of an annular flange on the distal end of a cartridge. The annular projections provide a tortuous path between the cartridge and the hub body thereby providing a sterile seal.

According an aspect of the present invention there is provided a medicament packaging assembly comprising a medicament delivery assembly as described in any paragraph previously and a cartridge having a medicament chamber for containing a medicament.

In the first configuration the proximal end of the cannula is spaced apart from a sealing member of the cartridge and in the third configuration the proximal end of the cannula has passed through the sealing member of the cartridge.

Optionally the medicament packaging assembly includes a needle shield. The needle shield may mount between the central hub portion and the engagement means. This helps to prevent inadvertent inward flexion of the resilient members and the cannula piercing the septum when it isn't wanted. The needle shield may be mounted using an interference fit to seal the distal end of the cannula between the needle shield and needle hub.

The needle shield may further comprising a barbed distal end to aid a user in removing the needle shield.

The needle shield and the hub body are provided with complementary features, for example a projection and aperture, to temporarily maintain engagement and reduce the chance of accidental removal of the needle shield.

### BRIEF DESCRIPTION OF THE DRAWINGS

One or more embodiments of the invention will now be described, by way of example only, with reference to the accompanying drawings, in which:

FIG. 1 shows a medicament packaging assembly;

FIG. 2 shows a needle hub according to the present invention;

FIGS. 3a and 3b show a body according to the present invention;

FIG. 4 shows a needle shield according to the presentation invention;

FIG. 5 shows a medicament packaging assembly in the first configuration;

FIG. 5 shows a medicament packaging assembly in the second configuration;

FIG. 5 shows a medicament packaging assembly in the third configuration.

### DETAILED DESCRIPTION

Certain terminology is used in the following description for convenience only and is not limiting. The word “distal” refers to the front, or patient, end of the medicament packaging. The word “proximal” refers to the rear end of the medicament packaging. The term “longitudinal”, with or without axis, refers to a direction on an axis through the device in the direction of the longest extension of the device. The term “radial” or “transverse/transversal”, with or without axis, refers to a direction generally perpendicular to the longitudinal direction, e.g. “radially outward” would refer to a direction pointing away from the longitudinal axis.

Medicament packaging 10 including a medicament delivery assembly 20 according to the present invention is illustrated in FIG. 1.

The medicament packaging assembly 10 includes a cartridge 102 having a medicament chamber 104 for containing a medicament. The cartridge 102 is generally tubular and has a reduced-diameter neck 106 at its distal end. An annular collar 108 extends around the neck 106. A piston member comprising a stopper or bung 110 is inserted into the proximal end of cartridge 102 to close the proximal end of the medicament chamber 104.

The distal end of the cartridge 102 is closed by a sealing element in the form of a disc-shaped elastomeric septum 112. The septum 112 is attached to the cartridge 102, for example, by a crimp fitting (not shown) comprising a crimp ring, preferably formed of a ductile metal such as aluminium. During assembly, the tubular crimp ring is mounted over the skirt and the ends of the ring are bent inwardly around the proximal side of the collar 108. The crimp ring when in place, applies a clamping force to the septum 112 to seal the septum 112 against the distal end of the cartridge 102.

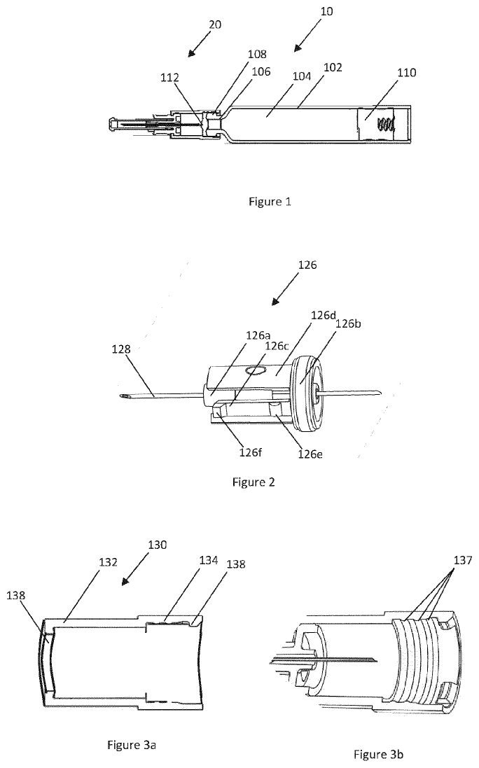

The medicament delivery assembly 20 comprises a hub 120, a body 130 and a needle shield 140 which will now be described in more detail with reference to FIGS. 2 to 4.

FIG. 2 illustrates the hub 120 comprises a hub body 126 and a cannula in the form of a double-ended hypodermic needle 128. The cannula is mounted through a central aperture in the hub body 126 through any suitable means, for example, adhesive, an interference fit or insert moulding.

The hub body 126 comprises a generally tubular central part 126a and a bell-shaped proximal part 126b. Needle shield retaining parts 126d extend distally from the proximal part 126b and is spaced apart from the central part 126a. Two further projections 126c extend distally from the proximal part 126b. The further projections 126c are preferably spaced apart from the central part 126a by the same distance as needle shield retaining parts 126d.

The further projections 126c are provided with two extensions 126e and 126f that extend outwardly away from the central axis of the hub body 126. The first extension 126e is shown as having a hemi-circular shape and the second extension 126f a flat distal face; however the skilled person will understand that any suitable shape may be used.

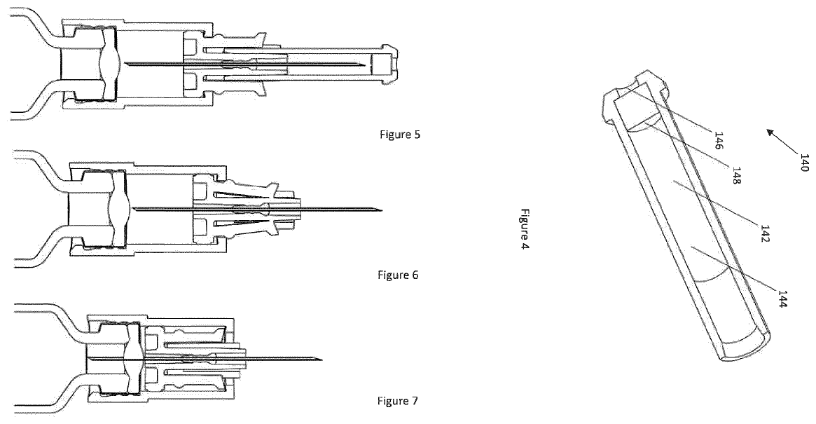

The hub body is configured to be received within a body 130 as illustrated in FIGS. 3a and 3b. The body 130 has a generally tubular cross-section with a distal portion 132 with a smaller diameter than a proximal portion 134. The proximal portion 134 of the body 130 is provided with an inwardly projecting annular flange 136 around its proximal end. The proximal portion 134 is configured to receive the cartridge 102.

The annular collar 108 of the cartridge is received within the proximal portion 134 with the inwardly projecting annular flange 136 is positioned proximally to the annular collar 108 of the cartridge 102 extending around the reduced-diameter neck 106. The proximal portion 134 is provided with a plurality of rings 137 projecting inwardly from the inner surface of the proximal portion. The plurality of rings 137 interfere with the outside diameter of the crimp of the cartridge to form a tortuous path sterile barrier.

The distal portion 132 is provided with an inwardly projecting annular flange 138 around its distal end. The interaction between the hub body 126 and body 130 will be described in more detail with reference to FIGS. 5 to 7 below.

A needle shield 140 configured to be fitted to the distal side of the hub body 126. The needle shield 140 has a distally-extending bore 142 that defines, in part, a cap chamber 144 or cannula chamber for receiving the distal end part of the needle 128.

A small-diameter bore 146 extends proximally from the distal end of the cap sleeve member 140 to connect with the distally-extending bore 142. The small-diameter bore 146 is plugged with a cylindrical closure 148 comprising an air-permeable sterile barrier material, such as are available under the registered trade marks Tyvek (DuPont, DE, USA) and Vyon (Porvair PLC., Norfolk, UK). The closure 148 allows air to flow into or out of the cap chamber 144 and the release member chamber 136 (through the flow path provided by the cannula 128) to equalise the pressure in the chambers 136, 144. This eliminates pressure gradients across the seals between the septum 112 and the coupling element 114, between the coupling element 114 and the hub body 126, and between the hub body 126 and the cap sleeve member 140, reducing the risk of failure of these seals. The sterile barrier properties of the material of the closure 148 prevent microbial contamination of the cap chamber 144. In some cases (not illustrated), where equalisation of pressure is not required, the small-diameter bore 146 may be omitted so that the cap chamber 144 is closed and sealed by the distal end of the cap sleeve member 140.

Assembly and use of the medicament delivery assembly will now be explained with reference to FIGS. 5 to 7.

FIG. 5 illustrates a medicament packaging assembly prior to use of the medicament packaging assembly. As can be seen the body 130 has received the hub body 126. In this, first configuration the distal inwardly projecting annular flange of the body 130 is situated between the bell-shaped proximal part 126b of the hub body 126 and the first extension 126e. The fit between the distal annular flange of the body and the hub is such that there is a seal. This may be achieved, for example by an interference fit. This is necessary to achieve a sterile volume between the hub body 126, body 130 and cartridge 102 in which the proximal end of the cannula is situated.

As discussed with reference to FIGS. 3a and 3b the annular collar 108 of the cartridge 102 is received within the proximal portion 134 of the body 130 with the inwardly projecting annular flange 136 is positioned proximally to the annular collar 108 of the cartridge 102 extending around the reduced-diameter neck 106. The proximal portion 134 is provided with a plurality of rings 137 projecting inwardly from the inner surface of the proximal portion. The plurality of rings 137 interfere with the outside diameter of the crimp of the cartridge to form a tortuous path sterile barrier.

Thus, provided the cartridge assembly 100 and the hub subassembly 124 are assembled in sterile conditions, the proximal end part of the needle 128 remains sterile until the the steps below occur.

The proximal end of the needle shield 140 is received within the space between the central part 126a of the hub body 126 the needle shield retaining parts 126d. To facilitate insertion the proximal end of the needle shield 140 wall may be tapered towards the proximal end. The needle shield 140 and hub body 126 when engaged, for example, through an interference fit form a sterile seal such that the distal end of the needle 128 remains sterile when the needle shield 140 and hub body 126 are engaged.

Optionally, the outside diameter of the shield and the needle shield retaining parts 126d have co-operating features, for example a projection and aperture to assist retaining the needle shield 140 within the hub body.

When the needle shield 140 is engaged with the hub body 126 the further projections 126c cannot move towards the central axis of the hub body 126.

FIG. 6 illustrates the medicament packaging assembly when the needle shield 140 has been removed from the hub body. As can be seen the removal of the needle shield 140 allows the further projections 126 to flex towards the central axis of the hub body 126. The flexing of the further projections 126 allows the first extension 126e and second extension 126f to pass through the aperture of the inwardly projecting annular flange 138 of the distal end of the body 130. The outer circumference of the needle shield retaining parts is equal to or less than the circumference of the aperture of the inwardly projecting annular flange 138 allowing the hub to move proximally towards the cartridge 102.

FIG. 7 illustrates the medicament packaging assembly in a third configuration when the hub body 126 has moved proximally such that the proximal end of the cannula 128 has passed through the septum such that medicament can flow through the cannula 128.

In the third configuration the further projections 126 have passed completely through the inwardly projecting annular flange 138. The proximal end of the needle hub is in contact with the distal end of the cartridge 102 and the distal end of the second extension 126f engages with the proximal side of the annular flange 138. This holds the cannula 128 in position when medicament is being delivered to the user.

It should be understood that the detailed description and specific examples are given by way of illustration only, since various changes and modifications within the spirit and scope of the disclosure will become apparent to those skilled in the art from the description.

1. A medicament delivery assembly for installation on a cartridge having a medicament chamber for containing a medicament; the medicament delivery assembly comprising:
a body for mounting on the cartridge;
a hub comprising engagement means moveable from a first configuration where the resilient member is held in engagement with the body and a second configuration where the hub is moveable relative to the body; and
a cannula mounted on the hub.

2. The medicament delivery assembly according to claim 1 wherein the engagement means is further moveable to a third configuration where the engagement means prevents movement relative to the body.

3. The medicament delivery assembly according to claim 1 wherein the engagement means comprises a resilient member;

4. The medicament delivery assembly according to claim 3 wherein the hub comprises a hub body and the engagement means comprises a projection extending distally from the hub body.

5. The medicament delivery assembly according to claim 4 wherein the distally extending projection further comprises a proximal outward extension spaced from the hub body and the distal end of the body comprises an annular flange which, in the first configuration is received between the hub body and the proximal outward extension.

6. The medicament delivery assembly according to claim 3, wherein the engagement means is further moveable to a third configuration where the engagement means prevents movement relative to the body, wherein in the third configuration the distal end of the resilient member engages with a proximal surface of an annular flange on the distal end of the body.

7. The medicament delivery assembly according to claim 6 wherein the distal end of the resilient member comprises an outward projection.

8. The medicament delivery assembly according to claim 1 wherein the hub includes a central portion through which the cannula is mounted.

9. The medicament delivery device as claimed in claim 8 wherein the engagement means are spaced away from the central portion.

10. The medicament delivery device as claimed in claim 9 wherein in the second configuration the resilient member is flexed towards the hub body.

11. The medicament delivery device as claimed in claim 1 wherein the body includes an inwardly extending annular flange on its proximal end.

12. The medicament delivery device as claimed in claim 1 wherein the body includes a proximal portion with a plurality of annular projections from its inner surface.

13. A medicament packaging assembly comprising a medicament delivery assembly as claimed in claim 1 and a cartridge having a medicament chamber for containing a medicament.

14. The medicament packaging assembly as claimed in claim 13 wherein, in the first configuration the proximal end of the cannula is spaced apart from a sealing member of the cartridge.

15. The medicament packaging assembly as claimed in claim 13 wherein in the third configuration the proximal end of the cannula has passed through the sealing member of the cartridge.

16. The medicament packaging assembly as claimed in claim 13 further comprising a needle shield.

17. The medicament packaging assembly as claimed in claim 16 wherein the engagement means are spaced away from the central portion and wherein the needle shield mounts between the central hub portion and the engagement means.

18. The medicament packaging assembly as claimed in claim 16 wherein the needle shield further comprising a barbed end.

19. The medicament packaging assembly as claimed in claim 16, wherein the needle shield and the hub body are provided with complementary features to temporarily maintain engagement.
